SEQUENCE LISTING (2 sequences)

SEQ ID NO: 1 - protein, 159 aa, mycobacterium
MKLTTMIKTAVAVVAMAAIATFAEPVALAAYPITGKLGSELTMTDTVGQVVLGWKVSDLKSSTAVIPGYPVAGQVWEATATVNAIRGSVTPAVSQFNARTADGINYRVLWQAAGPDTISGATIPQGEQSTGKIYFDVTGPSPTIVAMNNGMQDLLIWEP

SEQ ID NO: 2 - protein, 30 aa, mycobacterium
GQVWEATATVNAIRGSVTPAVSQFNARTAD

(19) United States
(12) Patent Application Publication
(10) Pub. No.: US 2014/0011735 A1
(43) Pub. Date: Jan. 9, 2014

(54) ANTIVIRAL COMPOSITION

(71) Applicants: Mendes, Fialho Arsenio Do Carmo Sales, Lisboa (PT); Filipe Santos Bernardes Nuno, Lisboa (PT); Braz Gonçalves João Manuel, Montijo (PT); Cunha Santos Ana Catarina, Lisboa (PT)

(72) Inventors: Mendes, Fialho Arsenio Do Carmo Sales, Lisboa (PT); Filipe Santos Bernardes Nuno, Lisboa (PT); Braz Gonçalves João Manuel, Montijo (PT); Cunha Santos Ana Catarina, Lisboa (PT)

(73) Assignee: AMRITA THERAPEUTICS LIMITED, Ahmedabad (IN)

(21) Appl. No.: 14/005,756

(22) PCT Filed: Mar. 5, 2012
(86) PCT No.: PCT/IN12/00153

§ 371 Date: Sep. 17, 2013

(30) Foreign Application Priority Data
Mar. 31, 2011 (IN) 1126/MUM/2011

(51) Int. Cl. C07K 14/35

(52) U.S. Cl. CPC C07K 14/35
USPC 514/3.8; 514/3.7; 514/4.3

(57) ABSTRACT

The present invention provides a composition comprising a broad spectrum protein of microbial origin as active anti-HIV/AIDS agent. Either the protein is secreted by or surface associated in microorganisms including but not limiting to bacteria, both pathogenic and non-pathogenic. The proteins used are isolated from bacteria Mycobacterium spp. specifically from Mycobacterium tuberculosis or M. bovis BCG. Further, the protein could be substituted by various truncated derivatives thereof, peptides derived from such proteins, synthetically prepared peptides, and proteins or peptides modified by PEGylation, acetylation, and phosphorylation. The protein includes purified proteins and peptides having amino acid sequence of SEQ ID No. 1 and 2 respectively.

### FIELD OF INVENTION

The present invention relates to biotherapeutics. The invention particularly provides pharmaceutical compositions containing an anti-viral agent useful for combating viral infections as an active therapeutic component and methods of application.

More particularly, the invention provides composition containing broad spectrum anti-HIV/AIDS agent, a protein of microbial origin. Specifically, either the protein is secreted by or surface associated in microorganisms including but not limiting to bacteria, both pathogenic and non-pathogenic.

Further, the proteins used are isolated from bacteria Mycobacterium spp. specifically from Mycobacterium tuberculosis or M. bovis BCG. The protein also can be substituted by various truncated derivatives thereof, peptides derived from such proteins, synthetically prepared peptides, and proteins or peptides modified by PEGylation, acetylation, and phosphorylation. The protein represents purified proteins and peptides.

The anti-HIV/AIDS agent of the present invention possesses enhanced efficacy and reduced toxicity. Further, the purified proteins and peptides employed as anti-HIV/AIDS agents, may have extended half-life and reduced immunogenecity in the patient blood stream.

The present invention also discloses the nature of the anti-HIV compound, pharmaceutical compositions and the manner of its applications as therapeutic agent to treat HIV/AIDS. The pharmaceutical composition comprises an active ingredient i.e. proteins, peptides, including PEGylated, acetylated, phosphorylated form thereof in isolation or in combination and physiologically and pharmaceutically accepted adjuvants or excipients. The proteins or peptides may be used in combination with other known anti-HIV/AIDS drugs. The proteins/peptides may have additional activity against other viruses such as polio, ebola, hepatitis B or C, dengue, influenza virus H1N1, herpes simplex, etc.

### BACKGROUND OF THE INVENTION

The Human Immunodeficiency Virus (HIV) is a highly pathogenic, evasive and difficult to eradicate agent that causes Acquired Immunodeficiency Syndrome (AIDS). There are two types of HIV, HIV-1 and HIV-2. Both types of HIV damage a person's body by destroying specific blood cells, called CD4+ T cells, which are crucial to helping the body's disease fighting ability. As per UNAIDS, 33.3 million people were living with HIV in 2009 (UNAIDS global report, 2010). This discovery in the early 1980s triggered major international scientific efforts in antiviral drug discovery and development. As a consequence, many drugs are now available to manage this condition, allowing the use of drug combination therapy known as HAART (highly active antiretroviral therapy). At present, 5 general classes of antiretroviral drugs have received FDA approval: nucleoside/nucleotide analog reverse transcriptase inhibitors (NRTIs), non-nucleoside analog reverse transcriptase inhibitors (NNRTIs), protease inhibitors (PIs), integrase inhibitors (IIs) and fusion inhibitors. The introduction of highly active antiretroviral therapy (HAART), a treatment paradigm using 3 or more antiretroviral drugs in combination, has led to significant declines in HIV-associated morbidity and mortality (Palella et al., 1998). Antiviral treatment options have primarily included combinations of two nucleoside analogue reverse transcriptase inhibitors (NRTI), and one protease inhibitor (PI). Alternative, preferred options include the use of two NRTIs with a non-nucleoside analogue reverse transcriptase inhibitor (NNRTI). Recently, NRTIs were combined with an integrase inhibitor for effective viral suppression and tolerability.

Over 25 years after the discovery of HIV as the etiologic agent of AIDS, no effective vaccine for the disease is available. This is because this disease agent is smart, and it quickly changes or masks the antigenic epitopes against the neutralizing antibodies that are generated in the body. Indeed, the two large-scale clinical trials (AIDSVax and STEP) aimed at testing proof-of-principle vaccines that were based on the induction of HIV-specific antibodies and cytotoxic T cell responses, respectively, failed to show any of the desired clinical efficacy (Fauci et al., 2008). A chronically replicating retrovirus HIV-1 presents some unusual challenges. The extensive genetic variation of HIV-1 is manifested by the numerous genetic subtypes worldwide and by the evolution of multiple viral variants within each infected individual. Previous exposure to most viral pathogens, either as the infectious agent or mimicked in a vaccine, results in the generation of antibodies that neutralize the virus and protect against disease (Stamatatos et al., 2009). Hence, a major goal of HIV-1 researchers is to design a vaccine that elicits protective antibodies in vivo that would be present before exposure to the virus (Burton et al., 2004). The isolation of HIV-1 in 1983 and the appreciation that neutralizing antibodies (NAbs) would probably target the envelope Env glycoprotein of this retrovirus led to optimism that an effective antibody-based vaccine could be designed within a few years. HIV-1 Env is a trimeric structure consisting of three identical gp160 molecules, each with a surface gp120 non-covalently linked to a membrane-spanning gp41 molecule, and, indeed, recombinant gp160 and gp120 vaccine candidates were rapidly produced and tested in phase 1 clinical trials. To the surprise of most researchers, these studies showed that the vaccine-induced antibodies failed their first in vitro test because they were unable to neutralize primary viruses derived from the blood of infected individuals (Mascola et al., 1996). Ultimately, the results of a phase 3 trial of a gp120 vaccine showed the lack of efficacy of this type of antibody-based vaccine strategy. The vaccine failed to prevent HIV-1 infection and did not lower viral replication or protect against CD4+ T cell decline (Flynn et al., 2005). Over the ensuing years, numerous attempts to design improved antibody-based vaccine immunogens met with limited success (Stamatatos et al., 2009).

Beyond viral replication or maturation inhibitors (NRTI, NNRTI, PI, II, etc), antiretroviral drugs so far have been based on targeting the virus attachment to CD4, its binding to cellular coreceptors CCR5 and CXCR4 and viral and host cell membrane fusion. Besides maraviroc, a recently approved CCR5 antagonist blocking gp120 co-receptor engagement (Fatkenheuer et al., 2005), enfuvirtide is the other only entry inhibitor approved for clinical use (Matthews et al., 2004). Enfuvirtide (also known as T-20) is a peptide drug selected from chemically synthesized peptides derived from various regions of gp41 (Wild et al., 1994). Enfuvirtide (T20) consists of a 36-amino acid synthetic peptide that is delivered as a subcutaneous injection. While shown highly effective in clinical trials, some patients develop painful and persistent injection-site reactions. Enfuvirtide works by competitively inhibiting interactions between the heptad repeat (HR) 1 and HR-2 domains of gp41, thus preventing the reconfiguration that allows the virus and cell surfaces to meet and fusion to occur (Kilby et al., 1998 and Rice and Wilantewicz, 2006). The same consortium that led enfuvirtide to clinical approval (Trimeris, Inc. and Roche) developed a second generation fusion inhibitor, T-1249. It is a 39-mer peptide which was designed taking into account the gp41 CHR sequences from HIV-1, HIV-2 and SIV (Simian Immunodeficiency Virus) (Eron et al., 2004). A successful short-term evaluation of antiretroviral activity and safety in humans proved the potential of this new drug (Eron et al., 2004), although further clinical development was put on hold (Martin-Carbonero, 2004). Since the first appearance of enfuvirtide, the search for peptide drugs against HIV has been a growing field of research and several candidates proved to be efficient in vitro (Naider and Anglister, 2009). A monoclonal antibody, TNX-355, that prevents the conformational change in gp120 needed to expose the coreceptor binding site, is given as weekly intravenous infusion. Despite such major advances in diagnosing and treating HIV infection, in 2009, 2.2 million new cases of AIDS were diagnosed in adults and 1.8 million deaths among people living with HIV were reported globally (UNAIDS global report, 2010).

The reasons why an effective drug or vaccine against AIDS is not available are complex and, for the most part, are related to specific features of lentiviruses and their interaction with the host immune system. Given the high mutability of the HIV-1 virus, leading to many sub-types, what is needed is a completely new approach to treat HIV-1. Such an approach, in addition to inhibiting viral growth, would block host functions that are critical for HIV-1 transport from the mucosal cell surface to the lymphatic T cells, and the viral entry. The need is for protein weapons with broad range of activity against viruses such as HIV-1, which can be obtained from pathogenic or non-pathogenic bacteria. Indeed, one such protein weapon, azurin, has been shown to have not only anticancer activity, but activity against viruses such as HIV/AIDS virus HIV-1 or the malarial parasite Plasmodium falciparum or toxoplasmosis-causing parasite Toxoplasma gondii (Chakrabarty 2010 & Fialho and Chakrabarty, 2010). Another protein, the ADI from Mycoplasma arginini, has been shown not only to have anticancer activity (Feun et al., 2010) but anti-viral activity against HIV-1 or hepatitis c virus (Kubo et al., 2006 and Izzo et al., 2007). Most importantly, however, azurin demonstrates strong growth inhibiting effect against three clades, European, Indian and African origin of HIV-1 virus (Chaudhari et al., 2006). Such strong growth inhibition (90% or greater) has been shown to be due to azurin's ability to interfere in the entry of HIV-1 to the host cells (Chaudhari et al., 2006). An azurin-like protein called Laz, is also produced by members of gonococci/meningococci such as Neisseria meningitidis that causes meningitis. Similar to 128 amino acid P. aeruginosa azurin, Laz has a 127 amino acid moiety highly homologous to P. aeruginosa azurin but has an additional 39 amino acid peptide in its N-terminal called an H-8 epitope. Laz is also highly efficient in strongly inhibiting not only the growth of cancers (Hong et al., 2006) and parasites (Chaudhari et al., 2006; Naguleswaran et al., 2008), but also the growth of HIV-1 virus (Chaudhari et al., 2006). The ability of azurin or Laz to strongly bind host proteins such as CD4, ICAM-3 or DC-SIGN, as well as the viral protein gp120, explains the growth suppressing ability of both azurin and Laz towards HIV-1. Unlike the protease, integrase, reverse-transcriptase or entry inhibitors developed by the pharmaceutical industry that inhibit only essential viral components, azurin not only strongly binds gp120 but also host proteins CD4, ICAM-3 or DC-SIGN that are important for HIV-1 transport and entry to the T cells. Blocking host functions will likely prevent HIV-1 to mutate to become drug resistant, since the virus cannot mutate to change the host proteins. It appears that P. aeruginosa designed its weapon azurin very cleverly not only to block the host apparatus for viral entry such as CD4 or ICAM-3, but also DC-SIGN to block the transport of HIV-1 from the mucosal surface to the T cells, thereby preventing infection.

We have recently demonstrated that the protein MPT63 secreted by Mycobacterium tuberculosis possesses both anticancer and anti-HIV/AIDS activity and filed a provisional patent application covering the anticancer activity (Suri et al., 2010).

After continued pain staking R & D work the inventor surprisingly found out totally unexpected hither to unknown property of the protein or variant thereof to combat viral infections particularly activity against HIV/AIDS Hepatitis B and C viruses, Dengue virus, Measles virus, Swine flu virus, Polio virus, Herpes simplex virus, Japanese Encephalitis virus, more specifically against HIV/AIDS.

MPT63 is a 159 amino acid (aa) small (16 kDa) protein which is secreted after 2-3 weeks of culturing. It consists of a 130 aa mature protein preceded by 29 aa signal peptide. This protein has been shown to have immunogenic property and has been implicated in virulence. It is specific to Mycobacteria as homologues of MPT63 have only been found in Mycobacterial species like M. smegmatis, M. bovis BCG and M. avium. A pseudogene of MPT63 has been found within the M. lapre genome, but is thought not to be translated into protein. The X-ray crystal structure of MPT63 was determined to 1.5-Angstrom resolution with the hope of yielding functional information about MPT63. The structure of MPT63 is a β-sandwich consisting of two antiparallel β-sheets similar to an immunoglobulin like fold, with an additional small, antiparallel β-sheet (Goulding et al., 2002). The function of MPT63 has hitherto been unknown and could not be predicted by its structural features as it has an extremely common immunoglobulin like fold that occurs in about 24% of the structures in the Protein Data Bank. The β-sandwich fold that MPT63 resembles is at the core of many proteins with diverse functions (Goulding et al., 2002). A 30 amino acid peptide, termed MB30 and derived from MPT63 protein, also possesses strong anticancer activity against a range of human cancers (Suri et al., 2010).

### OBJECT OF THE INVENTION

The main object is to provide an antiviral compositions eliminating the limitations of prior art. Particularly it provides anti-HIV/AIDS agent pharmaceutical compositions and methods of application thereof.

The other object is to provide an anti-HIV/AIDS agent, particularly broad spectrum anti-HIV/AIDS agent of microbial origin, more particularly proteins either secreted by or surface associated in microorganisms including but not limiting to bacteria, both pathogenic and non-pathogenic.

Yet another object is to provide purified proteins isolated from bacteria, specifically proteins isolated from Mycobacterium tuberculosis or M. bovis BCG useful as an anti-HIV/AIDS agent.

Still other object is to provide peptides derived from such proteins, synthetically prepared peptides, and proteins or peptides modified by PEGylation, acetylation, phosphorylation, etc. useful as an anti-HIV/AIDS agent.

Still another object is to also provide anti-HIV/AIDS agent comprising the proteins or various truncated derivatives thereof that possess enhanced efficacy and reduced toxicity.

Yet other object is to provide purified proteins and peptides, as an anti-HIV/AIDS agent, with extended half life and reduced immunogenecity in the patient blood stream. Such proteins and peptides may also be useful against other viruses such as dengue, polio, H1N1, hepatitis B and C, herpes, etc.

The other object of the present invention also discloses the nature of the anti-HIV compound, pharmaceutical compositions and the manner of its applications as therapeutic agent to treat HIV/AIDS. The pharmaceutical composition comprises an active ingredient i.e. proteins, peptides, including PEGylated, acetylated, phosphorylated form thereof in isolation or in combination and physiologically and pharmaceutically accepted adjuvants or excipients.

### STATEMENT OF INVENTION

Accordingly the present invention provides an antiviral composition comprising protein of amino acid sequence of SEQ ID NO: 1 or variant/truncated derivatives thereof and optionally suitable carriers and/or excipients.

According to one of the embodiments the protein may be purified protein isolated from micro-organisms or synthetically prepared.

Further, the protein may be secreted or surface associated and isolated from Mycobacterium spp.

The protein may preferably be obtained from Mycobacterium tuberculosis or M. bovis BCG.

The protein or variant thereof may further be modified by PEGylation, acetylation, phosphorylation wherein the variant may be peptides.

The peptides used may be having amino acids sequence of SEQ ID No. 2.

The present invention also provides a pharmaceutical composition comprising protein of amino acid sequence of SEQ ID NO. 1 or variant/truncated derivatives thereof having amino acid sequence of SEQ ID No. 2 as an active therapeutic component and pharmaceutically acceptable carriers and/or excipients in the range of 0.0 to 95% by wt.

The pharmaceutically acceptable carriers and/or excipients may be conventional one that facilitate the preparation of desired formulation and delivery of the active component by specified route. As such, the carriers and/or excipients used includes solvents, dispersion media, coatings antibacterial and antifungal agent isotonic and absorption delaying agents that are compatible with pharmaceutical administration and achieve required pharmacokinetics and pharmacodynamics of the active component. It may be noted that these carriers and/or excipients have to work interdependently or in synergy with the active component to deliver desired therapeutic effect.

The pharmaceutical composition of the present invention may be useful as intravenous (iv), intramuscular, oral, subcutaneous or topical application, against HIV/AIDS, Hepatitis B and C viruses, Dengue virus, Measles virus, Swine flu virus, Polio virus, Herpes simplex virus, Japanese Encephalitis virus.

The pharmaceutical composition as claimed in claim 7 is also useful for inhibition of virus propagation, blocking of gp-120 epitope.

SEQ ID NO.1:
MKLTTMIKTAVAVVAMAAIATFAEPVALAAYPITGKLGSELTMTDTVGQ
VVLGWKVSDLKSSTAVIPGYPVAGQVWEATATVNAIRGSVTPAVSQFNA
RTADGINYRVLWQAAGPDTISGATIPQGEQSTGKIYFDVTGPSPTIVAM
NNGMQDLLIWEP
SEQ ID NO. 2:
GQVWEATATVNAIRGSVTPAVSQFNARTAD (MB30)

Materials and Methods:

a. Selection of Protein for Anti-Viral Activity Preferably HIV/AIDS Activity

The complete amino acid sequence of MPT63 protein from Mycobacterium bovis or Mycobacterium tuberculosis is given below. The first 29 amino acids (underlined) in the following MPT63 sequence form secretion signal peptide (leader) sequence.

MKLTTMIKTAVAVVAMAAIATFAEPVALAAYPITGKLGSELTMTDTVGQ
VVLGWKVSDLKSSTAVIPGYPVAGQVWEATATVNAIRGSVTPAVSQFNA
RTADGINYRVLWQAAGPDTISGATIPQGEQSTGKIYFDVTGPSPTIVAM
NNGMQDLLIWEP

b. Cloning and Expression of MPT63 Gene

MPT63-encoding gene from Mycobacterium tuberculosis was amplified by PCR with genomic DNA as template. The forward and reverse primers used were: 5′-GCCTATCCCATCACCGGAAAA-3′ and 5′-CTACGGCTCCCAAATCAGCA 3′. The gene was placed downstream the T7 promoter in the pWH844 vector which also contained a 6×His fusion tag. E. coli SURE strain was used as a the host for expression in the following conditions: cells were incubated overnight in LB medium at 37° C. with 150 μg/ml of ampicillin and inoculated in the morning at an initial OD₆₄₀ of 0.1 in SB medium (3.2% tryptone, 2% yeast extract and 0.5% NaCl) with the same antibiotic concentration. When reached OD₆₄₀ 0.6-0.7, cells were induced with 0.2 mM IPTG and grown for 5-6 h at 37° C., 250 rpm. Cells were harvested by centrifugation at 8000 rpm for 10 minutes at 4° C., washed one time in buffer I (10 mM Imidazol, 0.2M mM sodium phosphate, 0.5M NaCl, pH 7.4), re-suspended in the same buffer and stored at −80° C. Cell disruption was performed by sonication and protein purification was performed in a histidine affinity chromatography column, HisTrap™ HP (GE Healthcare). Briefly, disrupted cells were centrifuged for 5 min, at 17600×g, 4° C., and the supernatant was centrifuged again at the same conditions for 1 h. The clarified extract was then loaded into a 5 ml HisTrap HP column equilibrated with START buffer (10 mM Imidazol, phosphate buffer: 0.2M sodium phosphate, 1M NaCl, pH 7.4) Protein elution was achieved with a continuous imidazole gradient (from 20 to 500 mM) in the same buffer. After purification, protein was immediately de-salted and buffer exchanged to PBS (137 mM NaCl, 2.7 mM KCl, 8.1 mM Na₂HPO₄.2H₂O, 1.76 mM KH₂PO₄, pH 7.4), pH 7.4, in HiPrep 26/10 Desalting column (GE Healthcare) in an AKTA purifier system following manufacturer instructions. Finally, protein was concentrated by centrifugation at 4° C. with Amicon Ultra Centrifugal Devices (Milipore) with a molecular mass cutoff of 10 kDa. Purified protein was passed through 1 ml Detoxi-Gel™ Endotoxin Removing column (Thermo Scientific) to remove endotoxins from E. coli host strain. At all steps, protein concentration was assessed with BCA™ Protein Assay kit (Thermo Scientific) following the manufacturer's instructions. The purity of protein was analyzed by sodium sulfate-polyacrylamide gel electrophoresis (SDS-PAGE).

c. Role of MPT63 Protein in Infection and Replication of HIV-1 NL4-3 in Primary Lymphocytes

CD4⁺ T-Cell Isolation:

Peripheral blood mononuclear cells (PBMCs) were isolated by Ficoll-Hypaque (Amersham BioSciences, Uppsala, Sweden) gradient centrifugation of leukopacks (Stanford Blood Bank, Stanford, Calif.) obtained by apheresis of healthy donors. CD4⁺ T cells were purified by negative selection using Microbeads (Miltenyi Biotec, Auburn, Calif.). Cell purity was determined by staining cells with fluorescently conjugated antibodies directed against CD4, CD3, CD8, and CD14 cells. Cell populations were found to be >95% CD3⁺CD4⁺.

CD4⁺ T-Cell Stimulation:

CD4⁺ T cells were activated by phytohemagglutinin (PHA) stimulation or by CD3/CD28 costimulation. For PHA stimulation, cells were cultured at a density of 2×10⁶ cells/ml with 1 μg of PHA (Sigma, St. Louis, Mo.)/ml for 24, 48, or 72 h. Cells were then washed to remove PHA and cultured for 48 h in RPMI 1640 medium (MediaTech, Herndon, Va.) supplemented with 15% fetal bovine serum (FBS) (Gemini, Woodland, Calif.) and 50 U of interleukin-2 (IL-2) (AIDS Research and Reference Reagent Program, Division of AIDS, National Institute of Allergy and Infectious Diseases [NIAID], National Institutes of Health [NIH])/ml. For CD3/CD28 costimulation, tissue culture plates were precoated with CD3 antibody. Briefly, wells were washed with 1× phosphate-buffered saline (PBS) and then coated with a 50 μg/ml stock solution of CD3 antibody. Excess liquid was removed, and plates were incubated at 37° C. until they gets dried. Cells were then cultured on coated plates at a concentration of 2×10⁶ cells/ml in the presence of 1 μg of soluble CD28 antibody (Becton Dickinson)/ml for 24, 48, or 72 h. Cells were removed from the CD3 coated plates, washed to remove soluble CD28, and then cultured in RPMI 1640 medium supplemented with 15% FBS and 50 U of IL-2/ml.

Viral Stocks:

HIV-1 NL4-3 stocks were prepared by transfecting 293T cells with pNL4-3. Lipofectamine 2000 (Invitrogen) was used for transfection according to the manufacturer's instructions. Essentially, lipid complexes were generated by mixing pNL4-3 and Lipofectamine 2000 in Optimem-I reduced serum medium (Invitrogen). 293T cells in Optimem-I (70 to 90% confluent) on poly-D-lysine-coated plates (Becton Dickinson) were incubated with lipid complexes for 5 h. The medium was changed to Optimem-I containing 10% heat-inactivated FBS. At 48 h posttransfection, the supernatant containing viral particles was harvested, clarified of cellular debris by centrifugation at 10,000×g for 10 min, and filtered through 0.22-μm-pore-size polyvinylidene difluoride membranes. Viral titers were determined by the p24 antigen enzyme-linked immunosorbent assay (ELISA) with a HIV-1 p24 ELISA kit (Innotest).

Cells were treated with MPT63 and subsequently infected with HIV-1 NL4-3. Cells were cultured in RPMI 1640 supplemented with 10% heat-inactivated FBS, minimal essential medium (MEM)-vitamins, nonessential amino acids, sodium pyruvate, 200 μM L-glutamine, 5.5×10⁻⁵ M β-mercaptoethanol, and 50 μg of gentamicin/ml. The cells were also supplemented with recombinant human interleukin 2 (50 U/ml; a generous gift of Hoffmann-La Roche, Nutley, N.J.). HIV-1 p24 antigen levels in cell extracts and culture supernatants were measured by an ELISA p24 antigen.

d. Role of MPT63 Protein in Infection and Replication of HIV-1 Primary Isolates of Clade G and C in CD4+ Lymphocytes

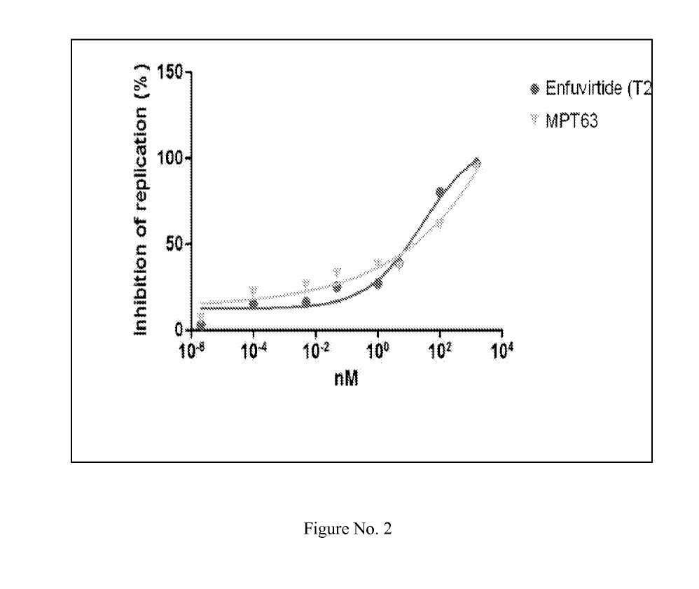

CD4+ Lymphocytes (10⁶) were preincubated, in duplicate, for 30 min with several concentrations of MPT63 protein as represented in FIG. 2 in Hanks' balanced salt solution prior to the addition of HIV-1 NL4-3. In pretreatment experiments, HIV-1 NL4-3 was preincubated with MPT63 protein for 30 min. Then MPT63-treated virus was added to T-cells (10⁶). After infection, the virus was removed after 1 h, and the cells were cultured for 5 days in presence of appropriate concentrations of MPT63. The negative control cells were incubated with the MPT63 diluents. Viral load was quantified using commercial p24 antigen ELISA (Innotest).

Virus Preparation:

The HIV-1 from clade G and C was isolated from infected patients and was kindly provided by Dr. Jose Miguel Pereira (Universidade Lisboa). Viral stocks were centrifuged at 1,000×g for 10 min to remove cell debris and then passed through a 45-μm-pore-size filter. The infectious titer of each viral preparation was determined by 50% tissue culture infective dose assay. Briefly, PHA-stimulated PBMCs from multiple donors were pooled and infected with serially diluted virus in quadruplicate wells. Cell supernatants were collected 5 days post infection, and HIV p24 antigen was quantified by p24 enzyme-linked immunosorbent assay (ELISA). Infections were scored positive for replication when p24 levels were higher than 50 pg/ml. The 50% tissue culture infective dose value represents the virus dilution at which 50% of wells scored positive for infection.

HIV Infection:

CD4⁺ T cells were activated by CD3/CD28 costimulation for 72 h prior to infection. Cells were treated with as described above, washed and incubated with virus at an MOI of 0.01 for 4 h at 37° C. After infection, cells were washed three times to remove any unbound virions and then cultured in RPMI 1640 medium supplemented with 15% FBS and 50 U of IL-2/ml.

Quantification of Viral Replication:

Viral replication was assessed by measuring the amount of soluble HIV p24 antigen in culture supernatants. Aliquots (200 μl) of supernatant were removed from infected cell cultures at 3, 5, 7, and 10 days post infection. Supernatants were stored at −80° C. until completion of the experiment. Quantification of p24 was determined using an ELISA (Innotest) according to the manufacturer's protocol.

e. Role of MPT63 in Cell-to-Cell Fusion

HIV Env-Mediated Cell Fusion.

The effect of MPT63 on HIV-1 Env-mediated cell fusion was analyzed with the previously described standard reporter gene activation assay (Schwartz et al., 1994). Briefly, effector cells were prepared by infecting HeLa cells in suspension with the recombinant vaccinia virus vCB-32 (encoding the HIV-1 Env SF162) and vP11T7gene1 (encoding the bacteriophage T7 RNA polymerase gene driven by a vaccinia virus promoter). Target cells were prepared by infecting HEK293-CCR5 cells with two recombinant vaccinia viruses, vCB21R-LacZ (encoding lacZ linked to the T7 promoter) and vCB-3 (encoding human CD4). Following overnight incubation at 37° C. to allow protein expression, effector and target cells were each washed and resuspended. Effector cells (100 μl, 2×10⁶ cells/ml) were added to duplicate wells of 96-well plates and preincubated for 15 min at room temperature with 10 μl of PBS containing different concentrations of MPT63. Target cells (100 μl, 2×10⁶ cells/ml) were then mixed with these effector cells. Effector cells were first incubated with MPT63 for 15 min at room temperature before mixing with the target cells. The cell mixtures were incubated for 2.5 h at 37° C. to allow fusion. The cells were then lysed with Nonidet P-40, and β-galactosidase (β-Gal) activity was measured at 570 nm in the presence of chlorophenol-red-β-D-galactopyranoside.

Enfuvirtide (T20), a competitive inhibitor of the heptad repeat (HR) 1 and HR-2 domains of gp41 was used as positive control in all the above experiments.

### DETAILED DESCRIPTION OF FIGURES

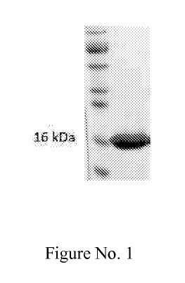

FIG. 1—depicts SDS-PAGE and Coomassie Brilliant Blue stain of MPT63 protein overproduced in E. coli SURE cells.

FIG. 2—depicts the comparative data of Inhibition of HIV-1 NL4-3 replication in peripheral blood lymphocytes by MPT63 protein and Enfuvirtide (T20). Peripheral blood lymphocytes were infected with HIV-1 NL4-3 in the presence of increasing concentrations of MPT63 protein (nM) with a multiplicity of infection of 0.01. Viral replication was assessed by measuring the amount of soluble HIV p24 antigen in culture supernatants at day 7. Quantification of p24 was determined using an ELISA and values represent mean values of triplicate samples Inhibition of replication was determined as percentage of p24 concentration relative to HIV-1 NL4-3 in the absence of MPT63.

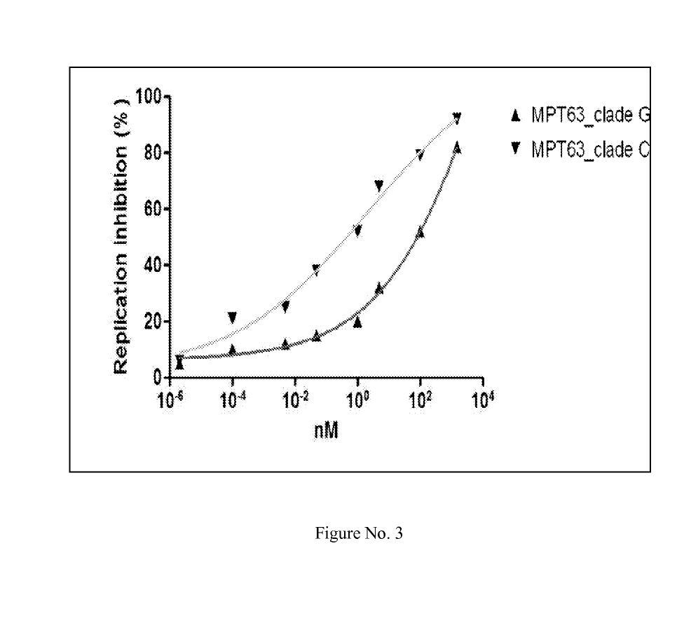

FIG. 3—depicts inhibition of replication of HIV-1 primary isolates clade G and C in CD4+ lymphocytes by MPT63 protein. Peripheral blood lymphocytes were infected with HIV-1 primary isolates from subtype G and C in the presence of increasing concentrations of MPT63 protein (nM) with a multiplicity of infection of 0.01. Viral replication was assessed by measuring the amount of soluble HIV p24 antigen in culture supernatants at day 7. Quantification of p24 was determined using an ELISA and values represent mean values of triplicate samples. Enfuvirtide (T20), a competitive inhibitor of the heptad repeat (HR) 1 and HR-2 domains of gp41 was used as positive control Inhibition of fusion was determined as the ratio of p24 concentration in the presence and absence of MPT63, considering that in the absence of MPT63 viral replication is 100%.

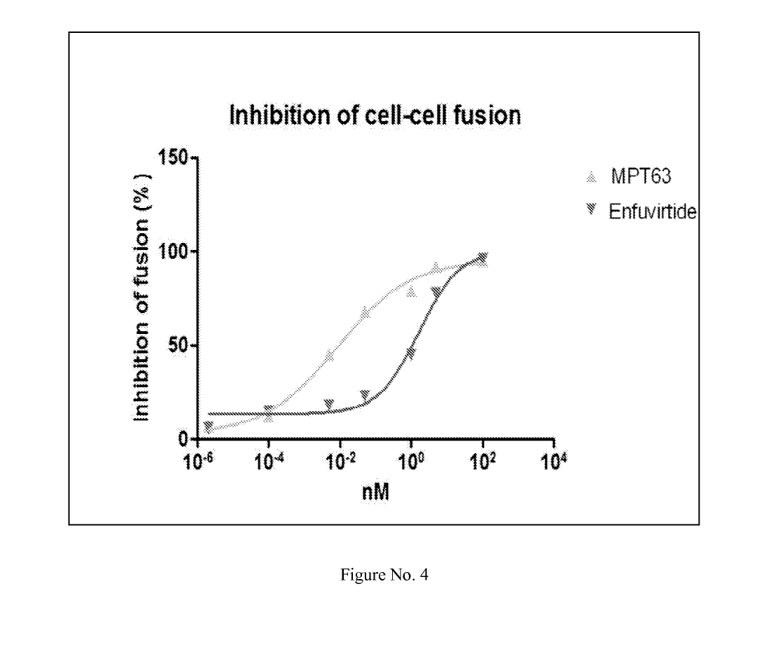

FIG. 4: depicts inhibition of cell to cell fusion by MPT63 protein. Effector HeLa cells expressing the Env glycoprotein and Tat were incubated with target HEK293 expressing CD4 and harboring LTR-β-Gal. Effector cells were added to target cells containing different concentrations of MPT63. After cell fusion the cell mixtures were disrupted in detergent and β-Gal activity was measured at 570 nm in the presence of chlorophenol-red-D-galactopyranoside. Enfuvirtide (T20), a competitive inhibitor of the heptad repeat (HR) 1 and HR-2 domains of gp41 was used as positive control. Inhibition of fusion was determined as the ratio of β-Gal activity in the presence and absence of MPT63, considering that in the absence of MPT63 cell fusion is 100%.

### DETAILED DESCRIPTION OF PREFERRED EMBODIMENTS

1. A method comprising administering to a patient who has HIV/AIDS a pharmaceutical composition consisting of one or more anti-HIV/AIDS compounds selected from the group consisting of MPT63 protein or various truncated derivatives of MPT63 protein, called peptides, all of which demonstrate anti-HIV/AIDS activity. The amino acid sequence of MPT63 protein is given under Materials and Methods. This compound may also be used in combination with other known anti-HIV/AIDS agents to enhance their potency.
2. The method of embodiment 1 where the viruses can be selected from the group consisting of all clades of HIV/AIDS virus. Of course, anti-viral activity of MPT63 protein, and peptides derived from it, can also be measured against other viruses such as Hepatitis B and C viruses, Dengue virus, Measles virus, Swine flu virus, Polio virus, Herpes simplex virus, Japanese Encephalitis virus and other viruses.
3. The method of embodiment 2 where the HIV/AIDS viruses are contacted by the bacterial protein or peptides leading to virus killing, inhibition of virus entry or fusion with host cells, inhibition of virus propagation, blocking of gp-120 epitope and other virus epitopes, blocking of host functions important in HIV/AIDS virus transport and/or entry into host T cells in HIV/AIDS patients or in other virus-infected patients.
4. A method whereby such proteins or peptides are introduced in patients infected with any of the viruses mentioned in embodiment 2.
5. The method of introduction of the proteins/peptides in virus patients may involve intravenous (iv), intramuscular, oral, subcutaneous or topical application, in presence or absence of adjuvants or excipients.
6. It is understood from common knowledge in protein chemistry that many of the amino acids can be replaced by other amino acids without loss of anti viral activity. Thus the protein or peptide sequences can be variable by 10 to 40% without any loss of activity.
7. The proteins or peptides in embodiment 1 wherein the structure of the protein or peptide can be modified by PEGylation, acetylation, phosphorylation, etc, to extend or optimize the half life of the protein or the peptide, or to reduce immunogenicity, in the patient bloodstream. Such modifications may also lead to extended virus targets other than HIV-1 such as polio, hepatitis B or C, dengue, H1N1, and others.

The MPT63 protein was purified to more than 95% purity as per the protocol described in materials and methods and further used in the experiments. Treatment of HIV/AIDS virus with MPT63 protein in this investigation revealed anti-HIV/AIDS property of the protein. All the experiments were carried out in triplicates and repeated three times.

As shown in FIG. 2, the replication of HIV-1 NL4-3 is competitively inhibited by MPT63 similar to Enfuvirtide (T20), with a EC50 of 10⁻² nM. The effect of MPT63 in a larger number of primary isolates of HIV/AIDS is further evaluated.

In order to check the effect of MPT63 protein in different clades of HIV/AIDS viruses, MPT63 protein was incubated with clade C and clade G of HIV/AIDS virus at the concentrations mentioned in materials and methods section. As shown in FIG. 3, the replication of primary isolates of clade C and G was competitively inhibited by MPT63 at various concentrations. The clade G of HIV-1 has shown more resistance to MPT63 protein than clade C primary isolate. Both the clades of viruses were effectively inhibited, with IC₅₀ ranging between 0.1 nM to 50 nM, thus revealing a considerable inhibition of replication of viruses by MPT63 protein depending on the HIV clade tested.

The effect of MPT63 on HIV-1 Env-mediated cell fusion was analyzed by reporter gene activation assay as described in materials and methods section. In this assay, cells expressing gp120 are targeted towards cell expressing CD4 and inhibition of this cell to cell fusion is monitored in presence of MPT63 protein. The FIG. 4 shows more inhibition of cell to cell fusion in presence of MPT63 protein as compared to the positive control Enfuvirtide (T20) indicating that viral transmission by cell contact could be highly inhibited.

All the above experiments have shown that MPT63 protein effectively inhibits the replication of HIV/AIDS virus as well as cell to cell transmission.

### REFERENCES

1. Burton D. R., Desrosiers R. C., Doms R. W., Koff W. C., Kwong P. D., Moore J. P., Nabel G. J., Sodroski J., Wilson I. A. and Wyatt R. T. 2004. HIV vaccine design and the neutralizing antibody problem. Nat. Immunol. 5: 233-236.
2. Chakrabarty A. M. 2010. Bioengineered bugs, drugs and contentious issues in patenting. Bioeng. Bugs. 1: 2-8.
3. Chaudhari A., Fialho A. M., Ratner D., Gupta P., Hong C. S., Kahali S., Yamada T., Haldar K., Murphy S., Cho W., Chauhan V. S., Das Gupta T. K. and Chakrabarty A. M. 2006. Azurin, Plasmodium falciparum malaria and HIV/AIDS: inhibition of parasitic and viral growth by azurin. Cell Cyc. 5: 1642-1648.
4. Eron J. J., Gulick R. M., Bartlett J. A., Merigan T., Arduino R., Kilby J. M., Yangco B., Diers A., Drobnes C., DeMasi R., Greenberg M., Melby T., Raskino C., Rusnak P., Zhang Y., Spence R. and Miralles G. D. 2004. Short-term safety and antiretroviral activity of T-1249, a second-generation fusion inhibitor of HIV. J. Infect. Dis. 189:1075-1083.
5. Fauci A. S., Johnston M. I., Dieffenbach C. W., Burton D. R., Hammer S. M., Hoxie J. A., Martin M., Overbaugh J., Watkins D. I., Mahmoud A. and Greene W. C. 2008. HIV vaccine research: the way forward. Science. 321: 530-532.
6. Fätkenheuer G., Pozniak A. L., Johnson M. A., Plettenberg A., Staszewski S., Hoepelman A. I., Saag M. S., Goebel F. D., Rockstroh J. K., Dezube B. J., Jenkins T. M., Medhurst C., Sullivan J. F., Ridgway C., Abel S., James I. T., Youle M. and van der Ryst E. 2005. Efficacy of short-term monotherapy with maraviroc, a new CCR5 antagonist, in patients infected with HIV-1. Nat. Med. 11:1170-1172.
7. Feun L., Kuo M. T., You M., Wu C. J., Wangpaichitr M. and Savaraj N. 2010. Arginine deiminase and cancer therapy. In Emerging Cancer Therapy: Microbial Approaches and Biotechnological Tools (A. M. Fialho and A. M. Chakrabarty, Eds). John Wiley & Sons, Hoboken, N.J. 199-217.
8. Fialho A. M. and Chakrabarty A. M. 2010. Promiscuous anticancer drugs from pathogenic bacteria: rational versus intelligent drug design. In Emerging Cancer Therapy Microbial Approaches and Biotechnological Tools (A. M. Fialho and A. M. Chakrabarty, Eds), John Wiley & Sons, Hoboken, N.J. 181-198.
9. Flynn N. M., Forthal D. N., Harro C. D., Judson F. N., Mayer K. H. and Para M. F. 2005. Placebo-controlled phase 3 trial of a recombinant glycoprotein 120 vaccine to prevent HIV-1 infection. J. Infect. Dis. 191: 654-665.
10. Global Report: UNAIDS Report on The Global AIDS Epidemic, 2010.
11. Goulding, C. W., Parseghian, A., Sawaya, M. R., Cascio, D., Apostol, M. I., Gennaro, M. L. and Eisenberg, D. 2002. Crystal structure of a major secreted protein of Mycobacterium tuberculosis—MPT63 at 1.5-Å resolution. Protein Sci. 11:2887-2893.
12. Hong C. S., Yamada T., Hashimoto W., Fialho A. M., Das Gupta T. K. and Chakrabarty A. M. 2006. Disrupting the entry barrier and attacking brain tumors: the role of the Neisseria H.8 epitope and the Laz protein. Cell Cyc. 5: 1633-1641.
13. Izzo F., Montella M., Orlando A. P., Nasti G., Beneduce G., Castello G., Cremona F., Ensor C. M., Holtzberg F. W., Bomalaski J. S., Clark M. A., Curley S. A., Orlando R., Scordino F. and Korba B. E. 2007. Pegylated arginine deiminase lowers hepatitis c viral titers and inhibits nitric oxide synthesis. J. Gastroenterol. Hepatol. 22: 86-91.
14. Kilby J. M., Hopkins S., Venetta T. M., DiMassimo B., Cloud G. A., Lee J. Y., Alldredge L., Hunter E., Lambert D., Bolognesi D., Matthews T., Johnson M. R., Nowak M. A., Shaw G. M. and Saag M. S. 1998. Potent suppression of HIV-1 replication in humans by T-20, a peptide inhibitor of gp41-mediated virus entry. Nat. Med. 4: 1302-1307.
15. Kubo M., Nishitsuji H., Kurihara K., Hayashi T., Masuda T. and Kannagi M. 2006. Suppression of human immunodeficiency virus type 1 replication by arginine deiminase of Mycoplasma arginini. J. Gen. Virol. 87: 1589-1593.
16. Martin-Carbonero L. 2004. Discontinuation of the clinical development of fusion inhibitor T-1249. AIDS Rev. 6: 61-61.
17. Mascola J. R., Snyder S. W., Weislow O. S., Belay S. M., Belshe R. B., Schwartz D. H., Clements M. L., Dolin R., Graham B. S., Gorse G. J., Keefer M. C., McElrath M. J., Walker M. C., Wagner K. F., McNeil J. G., McCutchan F. E. and Burke D. S. 1996. Immunization with envelope subunit vaccine products elicits neutralizing antibodies against laboratory-adapted but not primary isolates of human immunodeficiency virus type 1. J. Infect. Dis. 173: 340-348.
18. Matthews T., Salgo M., Greenberg M., Chung J., DeMasi R. and Bolognesi D. 2004. Enfuvirtide: the first therapy to inhibit the entry of HIV-1 into host CD4 lymphocytes. Nat. Rev. Drug Discov. 3: 215-225.
19. Naguleswaran A., Fialho A. M., Chaudhari A., Hong C. S., Chakrabarty A. M. and Sullivan W. J. Jr. 2008. Azurin-like protein blocks invasion of Toxoplasma gondii through potential interactions with parasite surface antigen SAG1. Antimicrob. Agents Chemotherap. 52: 402-408.
20. Naider F. and Anglister J. 2009. Peptides in the treatment of AIDS. Curr. Opin. Struct. Biol. 19: 473-482.
21. Palella F. J. Jr, Delaney K. M., Moorman A. C., Loveless M. O., Fuhrer J., Satten G. A., Aschman D. J. and Holmberg S. D. 1998. Declining morbidity and mortality among patients with advanced human immunodeficiency virus infection. N. Eng. J. Med. 338: 853-860.
22. Rice C. and Wilantewicz H. 2006. Fuzeon (enfuvirtide): efficacy, safety, patient acceptance, and strategies for managing injection-site reactions. AIDS Read. 16: 470-474.
23. Schwartz O., Alizon M., Heard J. M. and Danos O. 1994. Impairment of T cell receptor-dependent stimulation in CD4+ lymphocytes after contact with membrane-bound HIV-1 envelope glycoprotein. Virology. 198: 360-365.
24. Stamatatos L., Morris L., Burton D. R. and Mascola J. R. 2009. Neutralizing antibodies generated during natural HIV-1 infection: good news for an HIV-1 vaccine? Nat. Med. 15: 866-870.
25. Suri, A., Kanojia, D., Salunkhe, P., Surolia, A. and Chakrabarty, A. 2010. Anti Cancer Agent. Provisional patent application submitted to the Indian Patent Office on Oct. 1, 2010.
26. Wild C. T., Shugars D. C., Greenwell T. K., McDanal C. B. and Matthews T. J. 1994. Peptides corresponding to a predictive alpha-helical domain of human immunodeficiency virus type 1 gp41 are potent inhibitors of virus infection. Proc. Natl. Acad. Sci. USA. 91: 9770-9774.

1. An antiviral composition comprising protein of amino acid sequence of SEQ ID NO: 1 or variants or truncated derivatives thereof and pharmaceutically acceptable carriers and/or excipients in the range of 0.0% to 95.0% by wt.

2. The antiviral composition of claim 1, wherein the protein of SEQ ID NO: 1 is purified protein isolated from micro-organisms or synthetically prepared.

3. The antiviral composition of claim 2, wherein the protein is secreted or surface associated and isolated from Mycobacterium spp.

4. The antiviral composition of claim 3, wherein the Mycobacterium spp is Mycobacterium tuberculosis or M. bovis BCG.

5. The antiviral composition of claim 1, wherein the protein is modified by PEGylation, acetylation, or phosphorylation.

6. An antiviral composition comprising protein of amino acid sequence of SEQ ID NO: 2 or variants or truncated derivatives thereof, and pharmaceutically acceptable carriers and/or excipients in the range of 0.0% to 95.0% by wt.

7. A pharmaceutical composition comprising protein of amino acid sequence of SEQ ID NO: 1 or variants or truncated derivatives thereof and pharmaceutically acceptable carriers and/or excipients wherein the active therapeutic component is 0.1 to 50% by wt.

8. (canceled)

9. A method of inhibiting virus propagation in a patient suffering a viral infection, comprising administering to said patient a therapeutically effective amount of a protein of SEQ ID NO: 1 or variants or truncated derivatives thereof.

10. The method of claim 9, wherein the pharmaceutical composition is administered to said patient by intravenous (iv), intramuscular, oral, subcutaneous or topical application.

11. The method of claim 9, wherein the patient is suffering from a viral infection selected from the group consisting of: HIV/AIDS, Hepatitis B, Hepatitis C, Dengue virus, Measles virus, Swine flu virus, Polio virus, Herpes simplex virus, and Japanese Encephalitis virus.

12. A method of inhibiting virus propagation in a patient suffering HIV/AIDS infection, comprising administering to said patient a therapeutically effective amount of a protein of SEQ ID NO: 1 or variants or truncated derivatives thereof.
